(19) United States
(12) Patent Application Publication
(10) Pub. No.: US 2010/0028290 A1
(43) Pub. Date: Feb. 4, 2010

(54) DISINFECTANT

(76) Inventor: Drahomir Sokol, Ceska Skalice (CZ)

Correspondence Address: PETER R. RUZEK, 24 GREEN WOODS LANE, FARMINGTON, CT, 06085, US

(21) Appl. No.: 12/450,029

(22) PCT Filed: Mar. 5, 2008
(86) PCT No.: PCT/CZ2008/000025

§ 371 Date: Sep. 8, 2009

(30) Foreign Application Priority Data
Mar. 6, 2007 (CZ) PV2007-173

(51) Int. Cl. A61K 31/74

(52) U.S. Cl. 424/78.1

(57) ABSTRACT

A disinfectant, especially a skin disinfectant, an antiseptic agent for open wounds and a preparation for biocidal impregnation of plants, objects or materials, especially porous objects or materials containing cellulose, plastic or textile fibres, distinguishing itself by containing 10 to 99.9 mass percent of trichloroiodic(III) resin and 0.1 to 90 mass percent of an inorganic or organic active-chlorine-containing compound.

### TECHNICAL FIELD

This invention concerns a disinfectant which is especially suitable for skin disinfection, for antisepsis of open wounds and for biocidal impregnation of plants, objects or materials, especially porous objects and materials or those with the content of cellulose, plastic or textile fibres.

### BACKGROUND ART

A skin disinfectant should have a range of suitable properties such as the lowest possible toxicity, good tolerance by the organism even when used for a prolonged period of time, quick and safe effect on a wide range of microorganisms, a sufficient residual effect, no unpleasant odour, safe storage for a long time without any disintegration. There are many various skin disinfectants and preparations intended for antisepsis of open wounds.

The most commonly used disinfectants are alcohol- or water-soluble disinfectants. These disinfectants however are not the best ones as for their effectiveness. To inactivate microbial cells, it is necessary that the molecules or ions of the effective substance get into contact with the surface of the cell in a sufficient quantity. This is, however, prevented by the diffusion resistance of the laminar layer surrounding the cell which results in the necessity to use a sufficiently high concentration of the biocidal substance in the solution. In consequence of this high concentration, there can occur some undesirable side effects such as skin irritation, various allergies or eczemas. The concentration of the effective substance may even reach the threshold of toxicity. Biocidal preparations in the form of solution are also disadvantageous due to their application, storage and transportation at extreme thermal conditions. These preparations are more volatile at higher temperatures and their consistency may change at lower temperatures.

Generally, the most effective disinfectants are those based on active chlorine, bromine or iodine. Their rapid biocidal effect lies before all in halogenation of proteins. For that reason they are not suitable for skin disinfection in case there is a bleeding wound on it as the blood significantly decreases their biocidal effect.

CZ 295751 includes a description of an antibacterial preparation based on silver and/or silver halide on an inorganic carrier. The preparation contains 7.2 mass percent to 20 mass percent of silver and/or 15.7 to 25 mass percent of at least one silver halide. The rest is the inorganic carrier consisting of particles lower that 0.1 μm. Silver halide is a member of the group including silver bromide and iodide and the inorganic carrier consists of micro-particles of at least one compound that includes aluminum oxide, silicon dioxide, zinc oxide and titanium dioxide, aluminium hydroxide, magnesium carbonates and double magnesium carbonates, calcium carbonates and barium carbonates, lithium, sodium, potassium, magnesium, calcium and barium aluminosilicates and double aluminosilicates, and barium sulphate. The disadvantage of this preparation is the fact that it is available only in the form of powder which does not allow formation of a safe film on the treated surface.

Another disinfectant that is widely used is peracetic acid. Although this acid has excellent germicidal effects, it does not have any residual effects which means that the skin microflora renews in a short time after the initial inactivation. Besides, peracetic acid irritates the skin as it is an acidic and strongly oxidizing compound. Its concentrated solutions are explosive and diluted solutions disintegrate quickly.

Another well known and advanced sanitizing agents are the so-called iodophors in which iodine is, through micelles, bound to some high-molecular, water-soluble substances, e.g. polyvinylpyrolidone. Although iodine volatility in iodophors is relatively low it does not mean that there are no adverse effects on their residual action when used as skin disinfectants. For that reasons iodophors cannot be used at the temperatures exceeding 35° C. Moreover, iodine penetration into the skin should also be taken into account which means that frequent use of iodophors may result in various allergic reactions.

CS 185 097 also describes a disinfectant consisting of 10 to 99.9 mass percent of anioactive resin, e.g. polystyrenic polymer with benzyldimethylethanolammonium groups saturated with potassium triiodide and of 0.1 to 90 mass percent of ionogenic tenside, e.g. disodium salt of monoester of amidoethanol of lauric acid and sulfosuccinic acid and/or of non-ionogenic tenzide, e.g. polyethyleneglycoester providing always that the size of at least 80 per cent of the resin particles is up to 2 μm and the maximum size of the resin particles is up to 5 μm. The described skin disinfectant further consists of 5 to 90 mass percent of film-forming organic polymers, e.g. polyvinyl alcohol.

A disadvantage of this disinfectant is the fact that it is mostly used in its powder form or in the form of its water suspension which, in order to guarantee a certain antibacterial action, has to contain a relatively high amount of the active substance which may result in skin irritation. It is obvious from the description of that patent document that the authors' main effort was achieving an increased effectiveness my means of a great number of minimum-sized resin particles. This solution of the problem however leads to coagulation of the fine particles contained in the disinfectant into big aggregates resulting in quick follow-up sedimentation. Thus the biocidal effect of the disinfectant is significantly decreased.

Another generally-known disinfectant, according to CS 206 761, is a disinfectant based on iodochloridic anionic polystyrenic resin, i.e. a water suspension of this resin with an addition of a tenside and a small amount of a film-forming compound. This skin disinfectant and open wound antiseptic preparation contains 50 to 95 mass percent of anioactive resin where the functional group of this resin is saturated with iodine halogen derivatives, preferably with iodine trichloride in the amount of 5 to 50 mass percent provided always that the size of at least 80 mass percent of the resin particles is in the range of 0.2 to 2 μm and the maximum size of the particles ranges from 2 to 5 μm. The skin disinfectant further contains 0.1 to 90 mass percent of a ionogenic or non-ionogenic tenside, e.g. sodium salt of sulfosuccinic acid ester and fatty alcohol ethoxylate. The skin disinfectant may further contain 0.1 to 10 mass percent of a film-forming polymer, preferably polyvinyl alcohol.

This composition, however, does not enable the effective substance to penetrate the skin more deeply to hit a greater number of microorganisms. The consequence of this fact a decrease of the number of microorganisms per a time unit (e.g. 3 minutes) by three log. orders only which is not an optimum result. This can also be explained by the fact that the biocidal preparation caught on the skin gets dry very quickly. The absence of the water medium inhibits the transition of the biocidal ion from the anion resin onto the cell thus decreasing the time required for its inactivation. To ensure a higher effectiveness, a high concentration of the biocidal substance in the solution is needed which can lead to undesirable side effects in more sensitive patients such as skin irritation, allergies and eczemas. Another disadvantage of the disinfectant according to the CS 206 761 document is its low chemical stability. In water suspension, the anion of the chloroiodic(III) acid gets hydrolyzed with formation of hydrochloric acid by which the suspension significantly increases its acidity which further results in its increased irritability.

Moreover, by addition of the tensides, e.g. . the sodium salt of sulfosuccinic acid ester and fatty alcohol ethoxylate, this conception has not eliminated the disadvantage showing itself by coagulation of the fine particles in the disinfectant into big aggregates and the follow-up quick sedimentation described in CS 185 097. In this case as well, there was a necessity to increase the content of the active substance which was achieved by a great amount of very small particles of resin.

The goal of the invention is to create a non-toxic disinfectant that will be highly effective with a relatively small amount of the active substance in order to minimize the incidence of side effects such as skin irritation, various allergies and eczemas, that will be trouble-free as for its application, storage and transportation at extreme temperatures, that will be suitable for skin disinfection, for antisepsis of open wounds and for biocidal impregnation of objects and materials.

### DISCLOSURE OF THE INVENTION

The aforementioned weaknesses are, to a large extent, eliminated by the disinfectant, especially the skin disinfectant and preparation for antisepsis of open wounds, for biocidal impregnation of plants, objects or materials, especially porous materials and those with the content of cellulose, plastic or textile fibres, according to the invention the nature of which lies in the fact that it contains 10 to 99.9 mass percent of trichloroiodic(III) resin and 0.1 to 90 mass percent of an inorganic or organic active-chlorine-containing substance.

It is advantageous, if the active-chlorine-containing substance is sodium dichloroisocyanurate. Other compounds such as calcium hypochlorite, chloramine, chlorohexidine or magnesium hypochlorite may also be used.

By addition of the inorganic or organic active-chlorine-containing substance, the chemical stability of the trichloroiodic(III) resin is considerably increased which inhibits, especially in a water suspension, the formation of hydrochloric acid and the follow-up acidification of the suspension that leads to its higher irritability. The presence of the active chlorine shifts the reaction equilibrium of the system consisting of an anion of the trichloroiodic(III) acid and water in favour of the formation of iodine trichloride which has higher biocidal effects than monochlorine with its lower level of chlorination.

The disinfectant further contains 0.1 to 10 mass percent of an anion exchange resin with a medium-alkaline functional group in the OH form, preferably an anion exchange resin with a benzyldimethylethanolamionic-type medium-alkaline functional group.

By addition of the anion exchange resin with a medium-alkaline functional group in the OH form the chemical stability of the disinfectant is further increased. The anion exchange resin neutralizes the acid that may possibly be formed by which the optimum acidity of the disinfectant, i.e. the acidity ranging from pH 5 to pH 6, (even at extreme temperatures) is attained.

In addition to the above, it is recommendable that the disinfectant contains 10 to 90 mass percent of a gel-forming or film-forming components, preferably a mixture of a water solution of polyvinyl alcohol mixed with polyvinyl acetate, glycerol and polyethylene glycol.

It is also advantageous if the disinfectant further contains ethanol and/or isopropanol and/or polyvinyl pyrrolidone and/or paraffin derivatives and/or purified water.

In order to keep the optimum reaction within the range of pH 5 to pH 6, it is recommendable that the disinfectant further contains 0.1 to 5 mass percent of salts of weak acids, preferably calcium carbonate or magnesium carbonate.

It is further advantageous if the disinfectant contains 0.1 to 20 mass percent of a component having a curative effect on skin injuries. Such a component is e.g. salt of hyaluronic acid.

It is advantageous that the trichloroiodic(III) resin consists of polyvinyl styrene resin saturated with an anion of trichloroiodic(III) acid.

In case polymeric urethan or polyvinyl acrylate is used as a film-forming agent it is recommendable that the disinfectant further contains 0.1 to 10 mass percent of iron trichloride. Iron trichloride acts as a chlorine carrier and has oxidizing and condensing effects by which it stabilizes the trichloroiodic(III) resin, strengthens the film-forming effect of polymeric urethan and polyvinyl acrylate and their resistance against water hydrolysis.

The presence of the gel-forming components is essential in prevention of coagulation of the fine particles of the resin. Their effect lies in the fact that they form a thin layer on the surface of the particles which eliminates the action of adhesive forces between individual particles. Thus their agglomeration and the follow-up sedimentation are prevented. This allows the amount of the effective substance, the trichloroiodic(III) resin, to be considerably decreased which allows the disinfectant to be stored for a long time without decreasing its biocidal effects.

In addition, the presence of the gel-forming, anti-coagulation and film-forming ingredients having hydrophilic character guarantees the contact between the microbial cells and the surface of the biocidal particles in the disinfectant even if a bigger amount of water has evaporated.

The disinfectant under the invention thus considerably differs from the so far state-of-the-art disinfectants.

The invented disinfectant differs considerably from the other disinfectants known up to this day. The biocidal component of the invented disinfectant is an amount of very fine particles of polyvinyl styrene resin, the basic groups of which are saturated with an anion of trichloroiodic(III) acid in a relatively high concentration.

Trichloroiodic(III) acid can be prepared from various starting materials using three different chemical reactions, e.g. through reaction of metallic iodine with chlorine or reaction of potassium iodide and gaseous chlorine or chlorine generated by oxidation of hydrochloric acid. The optimum process appeared to be reaction between potassium iodide, hydrochloric acid and hydrogen peroxide.

KI+HCl=HI+KCl

2 HI+H₂O₂=I₂+2H₂O

6HCl+3 H₂O₂=3Cl₂+6 H₂O

I₂+3Cl₂=2ICl₃

2ICl₃+2 HCl=2 HICl₄

The solution of trichloroiodic(III) acid is light yellow and strongly acidic. Finely ground resin is then impregnated with this solution so that it contains optimally, related to the dry matter, 10 mass percent of ICl₄ and has light yellow colour.

The aforementioned acid, or more precisely its ICl₄ anion, although being a chlorine and iodine compound, does not have properties of halogens and its biocidal effect is not therefore based on halogenation of proteins. This is the reason why this preparation can be used for skin disinfection even if bleeding wounds are present as its effect is not inhibited by blood proteins.

The resin-bound trichloroiodic(III) acid is insoluble in water and it in fact does not passes into the water medium. When an indesirable microbial cell comes into contact with a particle of the biocidal resin, the chloroiodic(III) ion is quickly transferred onto this cell by which the cell is inactivated. The speed of this process has been verified on a wide range of microorganisms. Within a couple of dozen seconds, the original count of millions of harmful microorganisms in 1 ml falls to zero. It should be noted that the consumption of the biocidal anion is extremely low (trace amount)—one millionth of milligram per one cell.

In view of the above-mentioned strong bond to the resin, no penetration of ICl₄ into the body takes place. That is why no side effects such as skin irritation, allergies, etc. appear when this disinfectant is applied. The preparation is in no case toxic. As this disinfectant has also excellent virucidal effects it may become helpful in the prevention of spreading of dangerous viral infections.

The invented disinfectant has a considerably higher content of gel-forming, hydrophilic and film-forming components which, on the other hand, enabled the inventor to decrease the content of biocidal substance. In spite of this fact, its effectiveness compared with the present state-of-the-art disinfectants is considerably higher. After three-minute action the count of the active Escherichia coli cells falls down by 5 log. orders, the count of the Staphylococcus aureus cells by 4.5 to 5 log. orders. Similar situation is with Candida alb. as well as with a model virus. This high biocidal activity does not decrease even after 3-hour action. It has also been proven that even in this period of time no skin irritation or other undesirable side effects appear. The composition of this disinfectant according to this invention is, as it has already been mentioned above, stable which means that its biocidal effects are not decreased even after 7-year storage at the temperatures not exceeding 30° C.

The necessary amount of the disinfectant under this invention is several times lower if compared to liquid preparations. Its very important asset is its well-tested and verified antiseptic and germicidal action for at least 3 hours after application.

### DESCRIPTION OF EXAMPLES

### Example 1

First of all, using reaction between potassium iodide, hydrochloric acid and hydrogen peroxide, trichloroiodic(III) acid should be prepared.

KI+HCl=HI+KCl

2 HI+H₂O₂=I₂+2H₂O

6HCl+3 H₂O₂=3Cl₂+6 H₂O

I₂+3Cl₂=2ICl₃

2ICl₃+2 HCl=2 HICl₄

The solution of trichloroiodic(III) acid is light yellow and strongly acidic. An anion exchange resin is then impregnated with this solution to obtain trichloroiodic(III) resin containing, related to the dry matter, 10 mass percent of ICl₄.

Then, 99.4 mass percent of the formed finely ground trichloroiodic(III) resin is mixed with 0.6 mass percent of sodium dichloroisocyanurate. This mixture is then supplemented with 0.16 mass percent of finely ground anion exchange resin containing medium-alkaline benzyldimethylethanolamionic-type groups in the OH form. The result is a suspension of the aforementioned components with the trichloroiodic(III) resin.

To prepare the skin disinfectant, it is necessary to mix (intensive stirring is necessary) 18.6 mass percent of glycerol, 18.6 mass percent of polyethylene glycol, 11.7 mass percent of 17-per cent water solution of the mixture of polyvinyl alcohol and polyvinyl acetate, 8 mass percent of paraffin derivatives, and 4.8 mass percent of purified water. The formed gel-like mixture is then supplemented with 37.3 mass percent of the formerly prepared suspension of trichloroiodic(III) resin and 1 mass percent of calcium carbonate. The formed yellow-brown gel-like paste is easy to spread on the skin and has very good and long-term disinfecting effects.

### Example 2

First of all, as disclosed in Example 1, trichloroiodic(III) resin should be prepared.

Then, 98.7 mass percent of the formed finely ground trichloroiodic(III) resin is mixed with 1.3 mass percent of chloramine and 0.16 mass percent of finely ground anion exchange resin containing medium-alkaline benzyldimethylethanolamionic-type groups in the OH form is added to this mixture. The result is a suspension of the aforementioned components with the trichloroiodic(III) resin.

To prepare the skin disinfectant, it is necessary to mix (intensive stirring is necessary) 18.6 mass percent of glycerol, 18.6 mass percent of polyethylene glycol, 11.7 mass percent of 17-per cent water solution of the mixture of polyvinyl alcohol and polyvinyl acetate, 8 mass percent of paraffin derivatives, and 4.8 mass percent of ethanol. The formed gel-like mixture is then supplemented with 37.3 mass percent of the formerly prepared suspension of trichloroiodic(III) resin and 1 mass percent of magnesium carbonate. The formed yellow-brown gel-like paste is easy to spread on the skin, it gets dry quickly on the skin and has very good and long-term disinfecting effects.

### Example 3

First of all, as disclosed in Example 1, trichloroiodic(III) resin should be prepared.

Then, 98 mass percent of the formed finely ground trichloroiodic(III) resin is mixed with 2 mass percent of chlorhexidine and 0.16 mass percent of finely ground anion exchange resin containing medium-alkaline benzyldimethylethanolamionic-type groups in the OH form is added to this mixture. The result is a suspension of the aforementioned components with the trichloroiodic(III) resin.

To prepare the skin disinfectant, it is necessary to mix (intensive stirring is necessary) 15.6 mass percent of glycerol, 25 mass percent of polyethylene glycol, 15.6 mass percent of 17-per cent water solution of the mixture of polyvinyl alcohol and polyvinyl acetate, 12.5 mass percent of 2propanol. The formed gel-like mixture is then supplemented with 30.3 mass percent of the formerly prepared suspension of trichloroiodic(III) resin and 1 mass percent of calcium carbonate. The formed yellow-brown gel-like paste is easy to spread on the skin, it gets dry quickly on the skin and has very good and long-term disinfecting effects.

### Example 4

First of all, as disclosed in Example 1, trichloroiodic(III) resin should be prepared.

Then, 98 mass percent of the formed finely ground trichloroiodic(III) resin is mixed with 2 mass percent of chlorhexidine and 0.16 mass percent of finely ground anion exchange resin containing medium-alkaline benzyldimethylethanolamionic-type groups in the OH form is added to this mixture. The result is a suspension of the aforementioned components with the trichloroiodic(III) resin.

To prepare the disinfectant intended for treatment of varicose ulcers (ulcus crurris), it is necessary to homogenize (intensive stirring is necessary) 7.1 mass percent of glycerol, 34.2 mass percent of polyethylene glycol, 15.5 mass percent of 17-per cent water solution of the mixture of polyvinyl alcohol and polyvinyl acetate, 7.1 mass percent of polyvinyl pyrrolidone, 14 mass percent of the formerly prepared suspension of trichloroiodic(III) resin, 0.1 mass percent of calcium carbonate, 1 mass percent of calcium salt of hyaluronic acid, and 21 mass percent of purified water. The formed gel-like ointment can then be put on a gauze dressing in the amount 4 g per 1000 square centimetres and applied to the ulcer.

Alternatively, an absorptive non-woven fabric consisting of viscose, cellulose and plastic fibres can be impregnated with the ointment. A piece of this fabric with the area of 650 square centimetres and with the weight of 4.6 g containing 1.5 g of the ointment is then to be put to the ulcer and fixed with a bandage.

### Example 5

To prepare the disinfectant intended for preventive treatment and dry skin disinfection, e.g. of hands, it is necessary to put the mixture prepared according to Example 4 on an absorptive woven or non-woven fabric in the amount of 2 g per each 500 square centimetres of the fabric which already contains 10 g of purified water. The fabric or cloth prepared as described then can be used for treatment of the skin on the hands.

### Example 6

First of all, as disclosed in Example 1, trichloroiodic(III) resin should be prepared.

Then, 98.9 mass percent of the formed finely ground trichloroiodic(III) resin is mixed with 1.1 mass percent of magnesium hypochlorite and 0.16 mass percent of finely ground anion exchange resin containing medium-alkaline benzyldimethylethanolamionic-type groups in the OH form is added to this mixture. The result is a suspension of the aforementioned components with the trichloroiodic(III) resin.

To prepare the disinfectant intended for treatment of cold sores (herpes simplex), it is necessary to mix (intensive stirring at the temperature of 50° C. is necessary) 13.4 mass percent of glycerol, 40.2 mass percent of polyethylene glycol, 16.1 mass percent of 17-per cent water solution of the mixture of polyvinyl alcohol and polyvinyl acetate, 26.7 mass percent of the formerly prepared suspension of trichloroiodic(III) resin, 0.1 mass percent of calcium carbonate, and 3.5 mass percent of paraffin derivatives. The formed yellow solid paste is then formed into a lipstick to treat the cold sore.

### Example 7

First of all, as disclosed in Example 1, trichloroiodic(III) resin should be prepared.

Then, 98.7 mass percent of the formed finely ground trichloroiodic(III) resin is mixed with 1.3 mass percent of chloramine and 0.16 mass percent of finely ground anion exchange resin containing medium-alkaline benzyldimethylethanolamionic-type groups in the OH form is added to this mixture. The result is a suspension of the aforementioned components with the trichloroiodic(III) resin.

To prepare the skin disinfectant intended for treatment of mosquito or tick bites, it is necessary to mix 90 mass percent of the formerly prepared suspension of trichloroiodic(III) resin, 5 mass percent of polyethylene glycol, 3 mass percent of 17-per cent water solution of the mixture of polyvinyl alcohol and polyvinyl acetate, and 2 mass percent of 2-propanol. The formed yellow semisolid paste can then be rubbed into the bite or put on the tick.

### Example 8

First of all, as disclosed in Example 1, trichloroiodic(III) resin should be prepared.

Then, 99.4 mass percent of the formed finely ground trichloroiodic(III) resin is mixed with 0.6 mass percent of sodium dichloroisocyanurate. This mixture is then supplemented with 0.16 mass percent of finely ground anion exchange resin containing medium-alkaline benzyldimethylethanolamionic-type groups in the OH form. The result is a suspension of the aforementioned components with the trichloroiodic(III) resin.

To prepare the disinfectant intended for treatment of herpetic affections under the skin such as herpes zoster, it is necessary to homogenize (intensive stirring is necessary) 20 mass percent of glycerol, 20 mass percent of polyethylene glycol, 15 mass percent of 17-per cent water solution of the mixture of polyvinyl alcohol and polyvinyl acetate, 9.9 mass percent of the formerly prepared suspension of trichloroiodic(III) resin, 0.1 mass percent of calcium carbonate, 10 mass percent of 2-propanol, 15 mass percent of ethanol, 4 mass percent of paraffin derivatives, and 6 mass percent of purified water. The formed runny gel-like paste is to be rubbed into the affected place.

### Example 9

First of all, as disclosed in Example 1, trichloroiodic(III) resin should be prepared.

Then, 99.4 mass percent of the formed finely ground trichloroiodic(III) resin is mixed with 0.6 mass percent of calcium hypochlorite.

To prepare the disinfectant intended for biocidal protection of plants, various building materials such as wood, sandstone and sandstone sculptures, it is necessary to mix 50 mass percent of 17-per cent water solution of the mixture of polyvinyl alcohol and polyvinyl acetate with 50 mass percent of the formerly prepared mixture of trichloroiodic(III) resin and sodium dichloroisocyanurate.

The obtained solution is to be applied on plant or the aforementioned building material surfaces. If necessary, it can be further diluted with water.

### Example 10

First of all, as disclosed in Example 1, trichloroiodic(III) resin should be prepared.

Then, 98.4 mass percent of the formed finely ground trichloroiodic(III) resin is mixed with 0.6 mass percent of sodium dichloroisocyanurate and 1 mass percent of iron(III) chloride.

To prepare the disinfectant intended for biocidal treatment of insoles, it is necessary to mix 86.5 mass percent of water solution containing 28.6 mass percent of film-forming polyurethane polymers, 13.5 mass percent of the formerly prepared mixture of trichloroiodic(III) resin, sodium dichloroisocyanurate, and iron(III) chloride. The result is a water suspension by which the non-woven insole textile is to be impregnated.

### INDUSTRIAL UTILITY

The disinfectant under this invention can be used everywhere where there is a need for skin disinfection, for antisepsis of open wounds and for biocidal impregnation of plants, structures, objects or various other materials.

1. A disinfectant, especially a skin disinfectant, an antiseptic agent for open wounds and a preparation for biocidal impregnation of plants, objects or materials, especially porous objects or materials containing cellulose, plastic or textile fibres, comprising 10 to 99.9 mass percent of trichloroiodic(III) resin and 0.1 to 90 mass percent of an inorganic or organic active-chlorine-containing compound.

2. The disinfectant of claim 1, comprising 0.1 to 10 mass percent of anion exchange resin with medium-alkaline benzyldimethylethanol-amionic-type groups in the OH form.

3. The disinfectant of claim 1, comprising an additional content of 10 to 90 mass percent of gel-forming and film-forming components.

4. The disinfectant of claim 3, comprising 0.1 to 5 mass percent of weak acid salts to keep the optimum pH reaction within the range from pH 5 to pH 6.

5. The disinfectant of claim 4, wherein the weak acid salt is selected from the group consisting of calcium carbonate and magnesium carbonate.

6. The disinfectant of claim 1, and further comprising 0.1 to 20 mass percent of components to accelerate wound healing process.

7. The disinfectant of claim 6, wherein the wound-healing-process-accelerating component is a salt of hyaluronic acid.

8. The disinfectant of claim 1, wherein the active-chlorine-containing compound is selected from the group consisting of sodium dichloroisocyanurate, calcium hypochlorite, chloramine, chlorohexidine and magnesium hypochlorite.

9. The disinfectant of claim 1, wherein the trichloroiodic(III) resin consists of polyvinyl styrene resin saturated with an anion of trichloroiodic(III) acid.

10. The disinfectant of claim 1, and further comprising an additional content of 0.1 to 10 mass percent of iron(III) chloride.
